(19) United States
(12) Patent Application Publication
(10) Pub. No.: US 2023/0255542 A1
(43) Pub. Date: Aug. 17, 2023

(54) PATCH FOR SENSING A PHYSIOLOGICAL RESPONSE

(71) Applicant: West Pharmaceutical Services, Inc., Exton, PA (US)

(72) Inventors: Len T. MAGARA, King of Prussia, PA (US); Bingxi YAN, Downingtown, PA (US); Michael SULLIVAN, Birdsboro, PA (US); Alex LYNESS, West Chester, PA (US); Lawton E. LAURENCE, Chester Springs, PA (US); Madison SWOPE, Conneaut Lake, PA (US); Abby KOTWICK, Belmont, MA (US); Arjun MANOJ, Chihuahua (MX); John SAWICK, Mesa, AZ (US); Ron CHASE, Mesa, AZ (US); Craig BALDWIN, Chandler, AZ (US)

(21) Appl. No.: 18/109,801

(22) Filed: Feb. 14, 2023

Related U.S. Application Data
(60) Provisional application No. 63/310,446, filed on Feb. 15, 2022.

(51) Int. Cl. A61B 5/00; A61B 5/01; A61B 5/0531

(52) U.S. Cl. CPC A61B 5/411; A61B 5/01; A61B 5/0531; A61B 5/746; A61B 5/747; A61B 5/6833

(57) ABSTRACT

A patch for sensing distinct conditions indicative of a physiological response is described. The patch includes a base for attaching to a region of skin undergoing a physiological response. The patch also includes a first sensor provided on the base that detects a first condition indicative of the physiological response of the region of skin, a second sensor provided on the base that detects a second condition indicative of the physiological response, and a third sensor provided on the base that detects a third condition indicative of the physiological response. The first sensor, the second sensor, and the third sensor are each distinct sensor modalities that detect distinct conditions.

### CROSS REFERENCE TO RELATED APPLICATIONS

This application claims the benefit of U.S. Provisional Patent App. No. 63/310,446, filed Feb. 15, 2022, the disclosure of which is hereby incorporated by reference herein.

### TECHNICAL FIELD

This application generally relates to a patch for sensing a physiological response, and particularly relates to a patch for detecting distinct conditions indicative of the physiological response.

### BACKGROUND

Self-administered therapies, such as self-administered injections of various medications, can reduce the burden on the healthcare system and can provide greater control for patients in managing their own care. However, regulating patients as they self-administer the therapies can be a challenge. For example, patients sometimes decide to discontinue self-administered therapies before their treatment plan is finished, which can jeopardize treatment plans set forth by healthcare providers. Adverse events that occur in response to self-administered therapies, such as inflammation or swelling that occurs in response to injections, can be a factor that leads patients to decide to discontinue their self-administered therapies. Little is known about the types or the prevalence of these adverse events that occur in response to self-administered therapies. In addition, it would be advantageous for healthcare providers to receive tangible confirmation that self-administered therapies occurred as set forth in the treatment plans.

Accordingly, there exists a need for systems and devices for monitoring patients to detect the occurrence of a wide range of therapies, both self-administered and otherwise, and for tracking and quantifying the severity of adverse events that occur in response to these therapies.

### SUMMARY

These needs are met, to a great extent, by a patch for sensing conditions indicative of a physiological response. The patch includes a base configured to be attached to a region of skin undergoing the physiological response. The patch also includes a first sensor provided on the base that is configured to detect a first condition indicative of the physiological response of the region of skin, a second sensor provided on the base that is configured to detect a second condition indicative of the physiological response of the region of skin, and a third sensor provided on the base that is configured to detect a third condition indicative of the physiological response of the region of skin. The first sensor, the second sensor, and the third sensor are each distinct sensor modalities that detect distinct conditions.

In some aspects, at least one of the first sensor, the second sensor, and the third sensor may include two sensors disposed at different locations on the base. The first sensor may be a temperature sensor, and the first condition may be a temperature of the region of skin. The second sensor may be an impedance sensor, and the second condition may be an impedance of the region of skin. The third sensor may be an oxygen sensor, and the third condition may be an oxygen concentration of the region of skin. In some aspects, the oxygen may be tissue oxygen, blood oxygen, or both. The oxygen sensor may include a material that is phosphorescent in a presence of oxygen. Embodiments described herein can include any one of the features listed above or may include a combination of the above features. Implementations of the described techniques may include hardware, methods or processes, and/or computer software on a computer-accessible medium.

One general aspect includes a patch for sensing a temperature and an impedance indicative of a physiological response. The patch includes a base configured to be attached to a region of skin undergoing the physiological response. The patch also includes a temperature sensor provided on the base that is configured to detect the temperature of the region of skin that is indicative of the physiological response. The patch also includes an impedance sensor provided on the base that is configured to detect the impedance of the region of skin that is indicative of the physiological response.

In some aspects, the temperature sensor may include a first temperature sensor provided on an inner zone of the base and a second temperature sensor provided on an outer zone of the base extending radially from the inner zone of the base. The first sensor may be a digital integrated circuit sensor. The first sensor may include a Wheatstone bridge and a thermistor. The first sensor may include a resistive thermal difference element. The impedance sensor may include a first impedance sensor provided on an inner zone of the base and a second impedance sensor provided on an outer zone of the base extending radially from the inner zone of the base. The impedance sensor may include a wet electrode or a dry electrode. Embodiments described herein can include any one of the features listed above or may include a combination of the above features. Implementations of the described techniques may include hardware, methods or processes, and/or computer software on a computer-accessible medium.

Another general aspect includes a method for automatically determining an adverse reaction to a therapy. The method includes applying a patch to a patient. The patch may include a first sensor modality and a second sensor modality that are each configured to detect distinct conditions indicative of a physiological response of the patient. The method also includes administering the therapy to the patient at a position proximal to the patch. The method also includes sensing a condition of the distinct conditions indicative of the physiological response with at least one of the first sensor modality and the second sensor modality. The method also includes comparing the condition to a threshold. The method also includes determining, based on comparing of the condition to the threshold, the adverse reaction to the therapy.

Implementations may include one or more of the following features. In some aspects, the method may include evaluating, in response to determining the adverse reaction, a severity of the adverse reaction by comparing the condition to an elevated range of the threshold. The method may include determining, in response to comparing the condition to the elevated range of the threshold, that the adverse reaction is severe. The method may include automatically contacting emergency services in response to determining that the adverse reaction is severe. The method may include determining, in response to comparing the condition to the elevated range of the threshold, that the adverse reaction is not severe. The therapy may include a self-administered injection. In some aspects, the physiological response may include inflammation or swelling. Embodiments described herein can include any one of the features listed above or may include a combination of the above features. Implementations of the described techniques may include hardware, methods or processes, or computer software on a computer-accessible medium. The described embodiments may be configured to detect an adverse effect during an injection and may cause an action in response to the detection. The action may include an auditory, visual, and/or tactile notification to the user or medical professional. In some aspects, the action may include causing the injection process to stop, pause, restart, change speed, change injection pattern, or otherwise alter the injection mechanism. In such aspects, the patch may send a signal to the injection device, either wirelessly or through a wire, to cause the change in injection.

Various additional features and advantages of this invention will become apparent to those of ordinary skill in the art upon review of the following detailed description of the illustrative embodiments taken in conjunction with the accompanying drawings.

### BRIEF DESCRIPTION OF THE DRAWINGS

The following detailed description is better understood when read in conjunction with the appended drawings. For the purposes of illustration, examples are shown in the drawings; however, the subject matter is not limited to the specific elements and instrumentalities disclosed. In the drawings:

FIG. 1 shows a schematic view of a patch;

FIG. 2 shows a schematic view of a circular patch with first sensors;

FIG. 3 shows a schematic view of an oval patch with first sensors;

FIG. 4 shows a schematic view of a rectangular patch with first sensors;

FIG. 5 shows a schematic view of a patch with inkjet-printed second sensors;

FIG. 6 shows a schematic view of a patch with a plurality of circular second sensors;

FIG. 7 shows a schematic view of a patch with a plurality of second sensors in a triangular geometric arrangement;

FIG. 8 shows a schematic view of a patch with a plurality of second sensors in a geometric arrangement of concentric circles;

FIG. 9 shows a schematic view of a patch with a plurality of second sensors in pairs of parallel bars;

FIG. 10 shows a schematic view of a patch with a plurality of second sensors arranged in a dot pattern array;

FIG. 11 shows a schematic view of a patch with multiple distinct sensors according to a first embodiment;

FIG. 12 shows a schematic view of a patch with multiple distinct sensors according to a second embodiment;

FIG. 13 shows a schematic view of a patch with multiple distinct sensors according to a third embodiment;

FIG. 14 shows a schematic view of a patch with multiple distinct sensors according to a fourth embodiment;

FIG. 15 shows a schematic view of a patch with multiple distinct sensors according to a fifth embodiment;

FIG. 16 shows a process for automatically determining a presence of an adverse reaction to a therapy;

FIG. 17 shows a schematic view of a patch with multiple distinct sensors according to a sixth embodiment; and

FIG. 18 shows a cross-sectional view of a patch according to a seventh embodiment.

### DETAILED DESCRIPTION OF ILLUSTRATIVE EMBODIMENTS

This disclosure is directed to various embodiments of a patch that can sense distinct conditions indicative of a physiological response, such as erythema, inflammation or swelling of the skin, that can occur in response to a therapy (e.g., an injection of medicine) that is self-administered by the patient or administered by a healthcare provider. These patches can help healthcare provides monitor patients by detecting the occurrence of a wide range of therapies and by tracking and quantifying the severity of adverse events in response to such therapies.

The physiological response can occur, for example, as a normal response to a therapy (such as an injection), or can be indicative of an adverse reaction to the therapy. That is, the patches can provide a binary indication of whether the physiological response has occurred and/or can provide discrete measurements regarding the severity of the physiological response.

The patches of this disclosure can include a base that can be attached to a region of skin that may later undergo the physiological response. The patch can also include a first sensor provided on the base that can detect a first condition, such as a temperature, which can be indicative of the physiological response of the region of skin. In some embodiments, the patch can also include a second sensor provided on the base that can detect a second condition, such as an impedance, which can also be indicative of the physiological response of the region of skin. In some embodiments, the patch can also include a third sensor provided on the base that can detect a third condition, such as an oxygen concentration, which can also be indicative of the physiological response of the region of skin.

By sensing multiple distinct conditions indicative of the same physiological response, such as swelling, the patches of this disclosure can provide the patient and/or healthcare provider with an early warning of a possible adverse event. This early warning can provide valuable extra time for the patient to receive medical attention, which can include steps to mitigate the possible adverse event. In addition, since the patch can employ multiple sensors targeting distinct conditions indicative of the same physiological response, such redundancy in the patch can help detect the physiological response with superior accuracy relative to designs with a single sensor.

In addition, since the patches monitor physiological responses that can occur in response to many types of therapies (e.g., erythema, inflammation or swelling), the patches can be agnostic to therapy type. This allows the patches to be used in a wide range of applications. For example, the patches could be advantageous for monitoring injection sites after vaccine or allergy shot administration. Early detection of an adverse reaction at such injection sites could allow healthcare providers to treat patients and eliminate or mitigate against systemic reactions that can develop after the onset of an adverse reaction at the injection sites. Further, the patches can be advantageous for permitting patients to go about their day immediately after receiving vaccine or allergy shot injections since healthcare providers can be remotely alerted to any adverse reactions obviating the need for direct monitoring after the injections. The patches could additionally or alternatively be used in wound monitoring or as a complementary diagnostic tool.

In some embodiments, the patches can be used for monitoring instantaneous, or near-instantaneous, reactions caused by injections. This can include injections performed on a patient by a third part or self-injections, where the patient performs an injection on his or her own body. These patches can provide a patient or healthcare provider with feedback on whether or not an adverse event occurs by detecting measurements indicative of a physiological response, such as swelling, at the injection site.

In some embodiments, the patches can be used for longer term monitoring of adverse events. The patch can be configured to detect one or more conditions over a predetermined duration that can include an extended period of time (e.g., hours, days, weeks, or months) to allow for monitoring of a condition of the patient (e.g., chronic wound healing). For example, the patches could sense the temperature, hydration, impedance and/or tissue oxygenation at or around a wound. The sensed information can be used to determine a state of the wound and/or a presence of an infection. This feedback and gained insights would be invaluable to research and development efforts, device design, user experience, drug manufacturers, and patients alike. It would enable better decision making and more long-term views on the shifting landscape going from hospital and clinic settings to self-administered care in the home.

In some embodiments, the patches can be connected to a delivery device and can provide patient biometric data, such as heart rate, thus enabling feedback on drug or device effectiveness.

In some embodiments, the patches could be used together with an infusion set device with an extended wear soft cannula, which may remain attached after delivery. Such embodiments could utilize an off-body pump. This could allow the tubing to be disconnected post-delivery, and the patient's injection site could be monitored by the patch, which can remain adhered around the injection site. Alternatively, a pump could be directly built/integrated into the patch.

In some embodiments, the patch can be used as a standalone consumable skin contact sensor in conjunction with a syringe-based injection. The patch could be applied as part of a subcutaneous/intramuscular/intradermal infusion set, in which an off-body infusion device or syringe pump can infuse treatment through the infusion set into the patient's skin. In such concepts, a soft cannula or bent needle can be centered among sensors of the patch. The patch can be combined with an injector tip. Such a patch could be used, for example, in oncology, where a patient may inject a therapeutic, and the patch could be used to monitor the physiological response during and/or after the injection. Information received from this monitoring can be used to adjust treatment parameters, such as increasing or decreasing the dose of the therapeutic.

In some embodiments, the patch can be used as a companion diagnostic patch, where a nurse/healthcare professional may be in charge of caring for many patients at once (e.g., in a hospital, nursing home, clinic, or the like). That is, the patch can be used in short-term settings to monitor patients for adverse events or reactions in response to injections, receiving a medication, or another event that can cause a reaction.

In some embodiments, the patch could be used to help detect pregnancy. For example, the patch could monitor a patient using distinct sensing modalities, such as pH, skin hydration, and/or other combinations of biomarkers, to complement or provide an alternative to the traditional techniques of monitoring hCG and progesterone in urine samples.

In some embodiments, the patch can be used together with a wearable drug delivery device. The patch can be used with the wearable drug delivery device with minimal interruption or modification to the typical drug delivery process for the wearable drug delivery device. For example, the patch together with the wearable drug delivery device can be applied and worn for prescribed period as the drug is delivered via the wearable device. The patch in the wearable drug delivery device can be applied to the patient's body, for example, to an abdomen of the patient lateral to the naval. The patient could use the patch on areas of their bodies where multiple injections for chronic conditions have been performed. This would allow for monitoring the same area for conditions indicative of an adverse reaction in response to repetitive injections. This would significantly benefit patients with repeated injections in one area by providing objective feedback and insight into whether or not a site has an adverse event.

After the wearable drug delivery device delivers the drug, the wearable drug delivery device can be removed, and the patch can remain adhered to the patient. The patch can remain on the patient, and can detect, measure, and/or transmit data for a predetermined duration, which can last for a predetermined number of minutes, hours, or days. For example, in some aspects, the patch can be designed to remain adhered to the patient for up to 14 days after delivery of the drug. The patch can be worn while the patient engages in his or her normal activities throughout the day, which can include bathing, exercising, and sleeping. In such embodiments, the patch may be intended to monitor the desired site for adverse events for a longer duration after a medical treatment than in embodiments where the patch is designed to monitor for adverse events during or immediately after a medical treatment (e.g., an injection).

The patch can collect appropriate patient biometrics either continuously or at predetermined intervals and store them locally. The data can be transmitted from the patch to an external computing device or hub via one or more connection protocols, such as Bluetooth, Wi-Fi, NFC, cellular communication standards, and/or other suitable data transfer means. The data can be received and reviewed by a healthcare provider and used to guide future treatments or gauge the efficacy of treatments. This non-invasive monitoring provided by the patch can thus be seamlessly integrated into the drug delivery process and would provide value to patients, manufacturers, and quality/regulatory bodies alike.

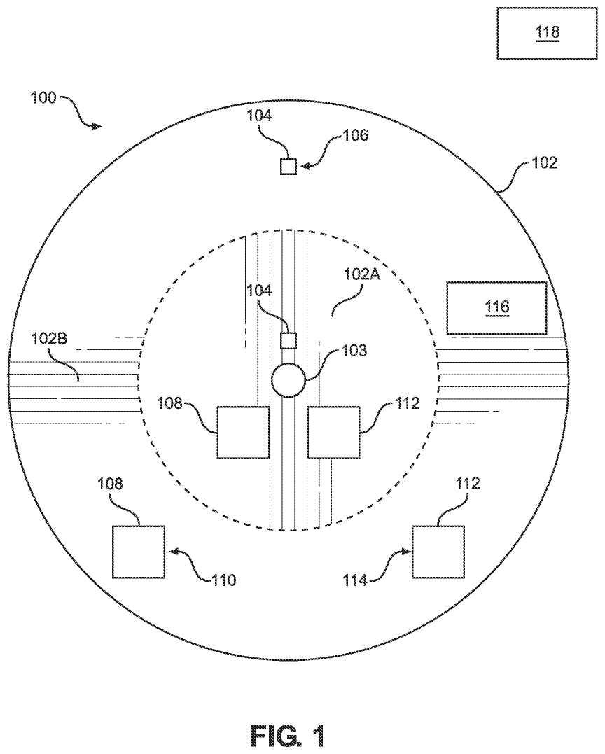

The above exemplary embodiments show that the patches of this disclosure have a myriad of uses within the healthcare and diagnostic spaces. The above features and uses can be incorporated into any embodiments of the patches and of processes involving the patches discussed in detail below in reference to FIGS. 1-18.

FIG. 1 illustrates a patch 100 for sensing conditions indicative of a physiological response according to aspects of the present invention. In an example embodiment discussed throughout this disclosure, the physiological response that is monitored can include swelling of a patient's skin, and the conditions being detected by the one or more sensors can includes conditions that are indicative of that swelling of the skin. It should be noted that this disclosure is not limited to swelling as the physiological response and contemplates other physiological responses as well.

Patch 100 can include a base 102. The base 102 can be attached to a patient at an area of interest (e.g., a region of skin) for monitoring physiological responses of the patient. The base 102 can include a hole 103 that can be arranged above a position within the area of interest. The hole 103 can provide external access to the position within the area of interest. For example, the hole 103 can provide a passage through base 102 for an injection of a medicine. The hole 103 can be located at any position on the base 102, such as at a central position of the base 102.

The patch 100 can include one or more sensors provided on and/or in the base 102. The sensors can detect and/or measure one or more conditions indicative of one or more physiological responses of the patient. In some embodiments, the patch 100 can include multiple sensors that sense distinct conditions indicative of the same physiological response (e.g., swelling). Distinct sensor modalities that can be sensed by the sensors of the patch 100 at the area of interest can include, for example, temperature, impedance, oxygen concentration, conductance, strain, hydration, biomarkers (e.g., cytokines), and/or other modalities. Since some conditions of the physiological response can manifest themselves more quickly than others, the ability to sense distinct sensor modalities can improve the speed at which a potential physiological response can be detected. Further, the ability to sense distinct sensor modalities introduces a redundancy in detection and can improve the accuracy of the physiological response detection by increasing the confidence that a physiological response has occurred. For example, if a patient wears the patch 100 in the shower, the patch 100 may record a temperature increase, which could be indicative of a physiological response to a therapy, or could simply be a physiological response to the temperature of the shower. However, since embodiments of the patch 100 can sense distinct sensor modalities in addition to temperature (e.g., impedance and/or oxygen concentration) the patch 100 can detect with greater accuracy that the physiological response is a response to the therapy if signals from other distinct sensor modalities are also received (i.e., if an impedance and/or oxygen concentration sensor similarly indicate presence of the condition).

In some embodiments, the patch 100 can include multiple sensors that sense the same condition indicative of a physiological response and that are disposed at different positions (e.g., within different zones) of the base 102, as will be discussed further below. This can provide the patch 100 with spatial data on the same condition, which can allow the migration of a physiological response to be tracked by patch 100. For example, the patch 100 can include an inner group of sensors that can be aligned in a first zone 102a (e.g., as indicated by the dense dotted region in FIG. 1) of base 102. First zone 102a can define an inner region of the base 102 proximal to an area of interest, such as an injection site. The patch 100 can include an outer group of sensors arranged in a second zone 102b (e.g., as indicated by the less-dense dotted region in FIG. 1) of base 102. Second zone 102b can define an outer region of the base 102 further away from the area of interest than the first zone 102a. At least some of the sensors provided in the inner group can sense the same conditions indicative of physiological response as at least some of the sensors in the outer group. Accordingly, a physiological response (e.g., swelling) originating at the area of interest can be monitored to determine if the physiological response spreads to the outer group of sensors arranged away from the area of interest. In some embodiments, the patch 100 can include various numbers of sensors or groups of sensor that can be aligned in a variety of zones. In addition, although the patch 100 can include multiple sensors that sense the same condition indicative of the same physiological response, in some embodiments, the patch 100 may include a single sensor for detecting one or more particular conditions indicative of a physiological response. This may be the case for embodiments where use of large sensors is required within a patch 100 that is too small to accommodate a plurality of such large sensors.

The base 102 can be provided in a variety of shapes and sizes. For example, the base 102 can be circular, square, triangular, irregularly shaped, or another suitable shape. In some embodiments, the base 102 can have a diameter and/or a maximum dimension that does not exceed a predetermined value. The predetermined value can depend on the intended use of the patch 100. For example, if the patch 100 is intended to be used on a small area of interest, the predetermined value may be smaller than if the patch 100 is intended to be used on a relatively larger area of interest. The predetermined value can be up to about 10 inches, up to about 5 inches, up to about 3 inches, or another suitable value. It will be appreciated that the exact predetermined value can deviate based on particular needs of the patch 100, manufacturing constraints, and/or other user requirements.

The base 102 can be attached to a patient using a variety of fixing techniques. For example, the base 102 can be attached to a patient via an adhesive. In some aspects, the adhesive may be applied to the base 102 directly, or, alternatively, to a separate layer (not shown) that is then adhered to the base 102 (e.g., double-sided tape). As another example, the base 102 can be attached to a patient using a strap or a belt. The base 102 can be fixed to the patient in a manner that prevents or limits fluid flow between the base 102 and the patient, which can improve the quality of certain sensor measurements, such as oxygen concentration.

In some embodiments, the base 102 can include a conformal flex circuit that can house the sensors and can allow for conformal electronics to be applied to an area on a patient's body. In some embodiments, such as those in which the patch 100 is used to monitor an injection, the conforming flex circuit can be applied to the abdomen or the torso, and the flexural properties of the conforming flex circuit can allow for even distribution of the sensors along the applied area. This can improve accuracy of readings via robust and secure contact with the patient's skin.

With continued reference to FIG. 1, the patch 100 can include a first sensor 104 that is can be configured to detect a first condition, such as a temperature, of the area of interest. A change in the first condition (e.g., a change in the temperature) can be indicative of a physiological response of the area of interest. For example, a rise in temperature of the area of interest detected by the first sensor 104 above a threshold temperature (e.g., from 0.5 degrees C. above the threshold to several degrees C. above the threshold, such as 1-4 degrees C.) can be indicative of severe inflammation of the area of interest. The first sensor 104 can be a digital integrated circuit sensor. The digital integrated circuit sensor can allow for simple data input/output and can minimize the need for additional components, such as communication boards. Digital integrated circuit sensors provide good sensitivity to various parameters that could be measured by the patch 100 and can thus improve the accuracy of temperature measurements indicative of the physiological response of the area of interest.

The first sensor 104 can include any of the following types of sensors: thermochromic liquid crystal sensors, voltage-diode temperature circuit sensors, infrared thermometers, thermal conductivity sensors, analog thermistors, etc. The first sensor 104 can include any number of temperature sensing elements. For example, sensing elements can include a Wheatstone bridge with the thermistor, an integrated circuit sensor, or a resistive thermal difference element. Integrated circuit sensors can be advantageous for their sensitivity. Resistive thermal difference elements can be advantageous for their wide operating range. In embodiments in which a Wheatstone bridge with a thermistor are used, the thermistor can be placed at a desired location on the patch 100 that would provide the most advantageous measurements, such as adjacent, or in proximity to, an injection site.

In some aspects, the patch 100 may include a plurality of first sensors 104. The patch 100 can include one, two, three, or more first sensors 104. In embodiments in which the patch 100 includes multiple first sensors 104, the first sensors 104 can be arranged at different spatial positions on the base 102 relative to each other or relative to other components of the patch 100. For example, the first sensors 104 can be arranged in a first geometric arrangement 106. Such arrangements can supply the patch 100 with spatial temperature data at different points in time. This data can be used to track aspects of the physiological response. For example, this data can be used to track the spread of swelling over time. As shown in exemplary FIG. 1, the patch 100 can include two first sensors 104—one provided in each of the first zone 102a and the second zone 102b of base 102. Although FIG. 1 depicts first zone 102a and second zone 102b, the first sensors 104 can each be arranged in any suitable number of zones of the patch 100. The first geometric arrangement 106 can include any suitable arrangement of the first sensors 104 relative to one another, such as 1 by 1, 2 by 1, 3 by 1, 3 by 2, 3 by 3, or another suitable arrangement. The first geometric arrangement 106 may be disposed adjacent to, or relative to, a geometric center of the base 102. In some aspects, the base 102 may define a hole 103 extending through the base 102. The hole 103 may be defined at, or adjacent to, the geometric center of the base 102. The first geometric arrangement 106 may be disposed radially around the hole 103. In some aspects, the hole 103 may allow for a needle to pass through the patch 100 to contact the patient's skin to perform the injection.

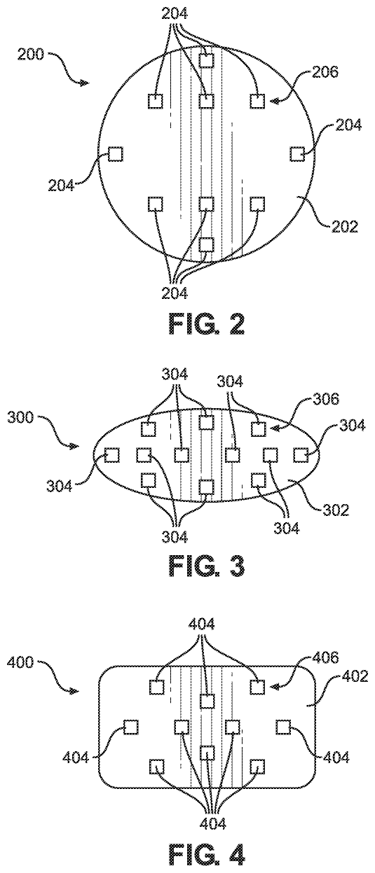

FIGS. 2, 3, and 4 show patches 200, 300, and 400, respectively, each having multiple first sensors 204, 304, or 404 arranged in respective first geometric arrangements 206, 306, and 406. For example, as shown in FIG. 2, the patch 200 can include a circular base 202 and a roughly circular first geometric arrangement 206 that includes a plurality of the first sensors 204. In some embodiments, the patch 200 with circular base 202 can include any number of other first geometric arrangement 206 arrangements, including groupings of first sensors 204 in different quartered zones of the circular base 202.

As shown in FIG. 3, the patch 300 can include an oval shaped base 302 having a first geometric arrangement 306 that includes a plurality of the first sensors 304. In some embodiments, other first geometric arrangements 306 can be provided on a similarly shaped base 302.

As shown in FIG. 4, the patch 400 can include a rectangular or shaped base 402 having a first geometric arrangement 406 that includes a plurality of the first sensors 404 as shown. In some embodiments, other first geometric arrangements 406 can be provided on a similarly shaped base 402.

The patches 200, 300, and/or 400 can include any of the features of any of the previously described patches, and vice versa. In addition, although patches 200, 300, and 400 show geometric arrangements of first sensors, other embodiments of this disclosure can include similar geometric arrangements on similarly shaped bases for other sensors discussed further (e.g., second sensors, third sensors, etc.) either in addition to or instead of the first sensors.

Returning to FIG. 1, another of the sensors that can be provided with patch 100 is a second sensor 108 that can detect a second condition of the area of interest. In some examples, the second condition can include an impedance of the skin. A change in impedance can be indicative of a physiological response of the area of interest. For example, a decrease in the impedance of the area of interest detected by the second sensor 108 relative to a threshold impedance (e.g., below 6 k ohm together with a decline in a phases angle of for example −10 degrees) can be indicative of inflammation or swelling of the area of interest. The second sensor 108 can be configured to measure impedance at frequencies between 100 Hz and 50 kHz. The second sensor 108 can include wet and/or dry electrodes. Wet electrodes can include a conductive gel. In some aspects, wet electrodes can include gold-plated electrodes embedded in a conductive gel and can include a ring with a thermal coefficient of expansion similar to that of the gold-plated electrodes. Wet electrodes can be advantageous due to their manufacturability, non-invasive and conforming attachment to the skin, and accuracy. Dry electrodes can include microneedles. Dry electrodes can be advantageous due to their ability to probe and measure below the surface of the skin, penetrating the electrically resistive stratum corneum and delivering current directly into epidermis layers. In some aspects, a wet electrode may result in less background noise when on the skin (compared to a dry electrode) due to its continuous moistening effect on skin. A wet electrode can be used with an adhesive layer of cation-enriched hydrogel, which can help reduce electric resistance. In some aspects, a dry electrode can provide higher sensitivity than a wet electrode. Embodiments disclosed herein can utilize a wet electrode, a dry electrode, or both a wet and dry electrode.

In some embodiments, the second sensor 108 can intermittently detect the second condition. For example, second sensor 108 can include a reader and/or an applicator. The reader and/or applicator can selectively open and close a circuit with the wet and/or dry electrodes to intermittently detect the second condition (e.g., impedance). According to this configuration, the wet and/or dry electrodes can remain in constant contact with the patient, but the current passing through the patient's tissue can be limited due to the intermittent opening and closing of the circuit. Alternatively, second sensor 108 can be configured to continuously detect the second condition.

In some aspects, the patch 100 may include a plurality of second sensors 108. The patch 100 can include any suitable number of second sensors 108, including one, two, three, or more second sensors 108. In embodiments in which the patch 100 includes multiple second sensors 108, the second sensors 108 can be arranged at different spatial positions on the base 102 relative to one another or relative to other components of the patch 100. For example, the second sensors 108 can be arranged in a second geometric arrangement 110. Such arrangements can supply the patch 100 with spatial impedance data at different points in time. This data can be used to track aspects of the physiological response. For example, this data can be used to track the spread of swelling over time. As shown in FIG. 1, the patch 100 can include two second sensors 108—one provided in each of the first zone 102a and the second zone 102b of base 102. The second sensors 108 can each be arranged in any suitable number of zones of the patch 100. The second geometric arrangement 110 can include any suitable arrangement of the second sensors 108 relative to one another, such as 1 by 1, 2 by 1, 3 by 1, 3 by 2, 3 by 3, or another suitable arrangement. The second geometric arrangement 110 may be disposed adjacent to, or relative to, the geometric center of the base 102, which may define a hole 103 as described above. The second geometric arrangement 110 may be disposed radially around the hole 103.

FIGS. 5, 6, 7, 8, 9, and 10 show patches 500, 600, 700, 800, and 1000, respectively, having multiple second sensors 508, 608, 708, 808, 908, and 1008 arranged in respective second geometric arrangements 510, 610, 710, 810, 910, and 1010. For example, as shown in FIG. 5, the patch 500 can include a circular base 502 and an inkjet-printed second geometric arrangement 510, which includes a plurality of the second sensors 508, having multiple branches with circular electrodes extending therefrom.

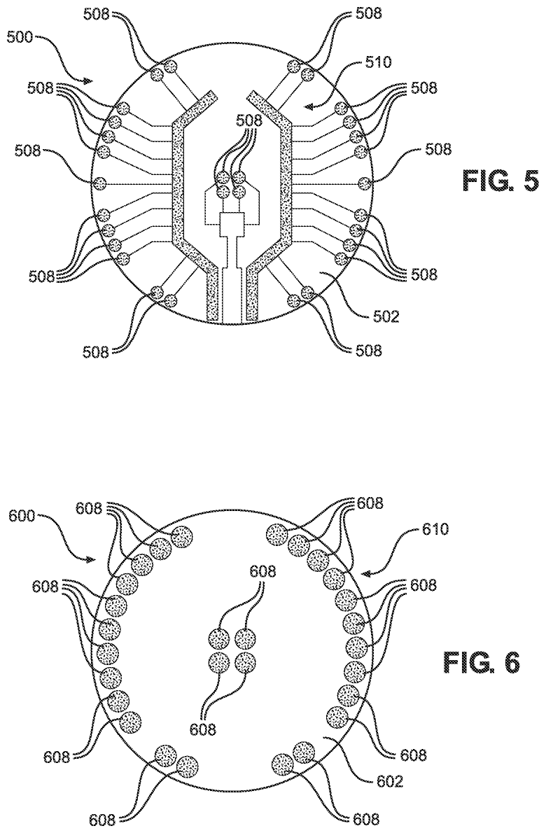

As shown in FIG. 6, the patch 600 can include a circular base 602 having a second geometric arrangement 610, which includes a plurality of second sensors 608, including a plurality of circular electrodes arranged around a circumference of circular base 602 and a plurality of circular electrodes arranged towards a center of circular base 602.

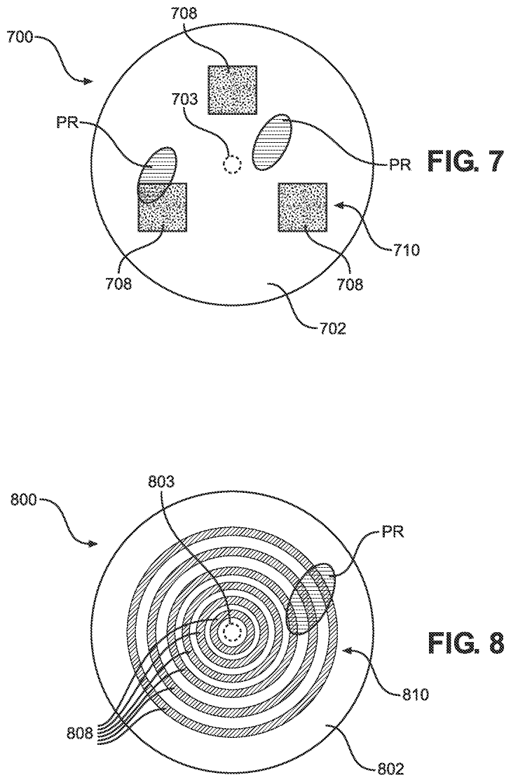

As shown in FIG. 7, the patch 700 can include a circular base 702 having a second geometric arrangement 710, which includes a plurality of the second sensors 708, provided in a triangular pattern that can be centered about a geometric center of the base 702. The geometric center of the base 702 can define a hole 703 through which a needle can be inserted during an injection. The second geometric arrangement 710 can be configured to sense a second condition from at least a portion of a physiological response PR, as shown.

As shown in FIG. 8, the patch 800 can include a circular base 802 having a second geometric arrangement 810, which includes a plurality of the second sensors 808, including a plurality of concentric circle electrodes that can be centered about the geometric center of the base 802, which may include a hole 803. The second geometric arrangement 810 can be configured to sense a second condition from at least a portion of the physiological response PR, as shown.

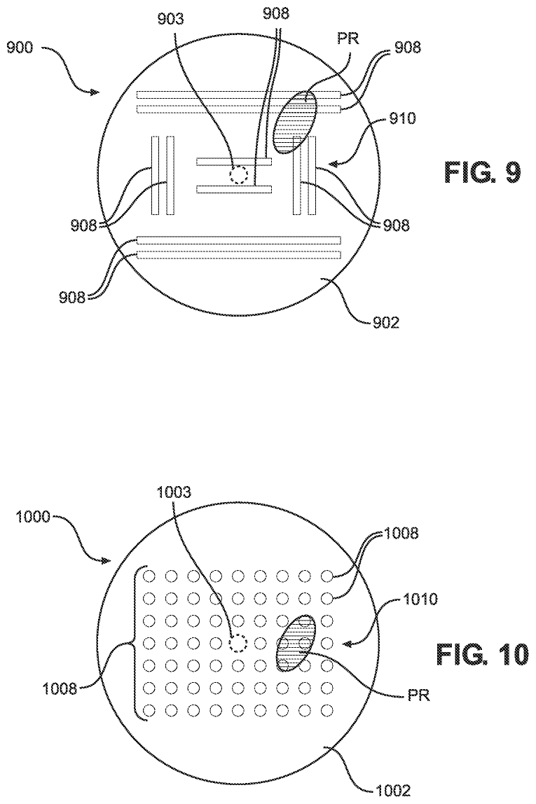

As shown in FIG. 9, the patch 900 can include a circular base 902 having a second geometric arrangement 910, which includes a plurality of the second sensors 908, that can include electrodes arranged in parallel bars that can be oriented about a geometric center of the base 902, which can include a hole 903. The second geometric arrangement 910 can be configured to sense a second condition from at least a portion of the physiological response PR, as shown.

As shown in FIG. 10, the patch 1000 can include a circular base 1002 having a second geometric arrangement 1010, which includes a plurality of the second sensors 1008, that can include electrodes arranged in a dot-patterned array that can be centered about a geometric center of the base 1002, which can include a hole 1003. The second geometric arrangement 1010 can be configured to sense a second condition from at least a portion of the physiological response PR, as shown.

Patches 500, 600, 700, 800, 900 and/or 1000 can include any of the features of any of the previously described patches, and vice versa. In addition, although patches 500, 600, 700, 800, 900 and 1000 show geometric arrangements of second sensors, other embodiments of this disclosure can include similar geometric arrangements on similarly shaped patches for other sensors discussed herein (e.g., first sensors, third sensors, etc.) either in addition to or instead of the second sensors.

Returning to FIG. 1, another of the sensors that can be provided with patch 100 is a third sensor 112 that can detect a third condition, such as oxygen concentration, of the area of interest. A change in oxygen concentration can be indicative of a physiological response of the area of interest. For example, an increase in oxygen concentration at the area of interest detected by the third sensor 112 relative to a threshold oxygen concentration can be indicative of swelling of the area of interest.

In some embodiments, the third sensor 112 can include an oxygen sensor that can be embedded within a film. The oxygen sensor can include a material, such as metalloporphyrin, that is phosphorescent in the presence of oxygen. Oxygen concentration can be determined based upon the degree of phosphorescence detected by the third sensor 112. Additionally, or alternatively, the third sensor 112 can include a pulse oximeter to detect a concentration of oxygen.

In some aspects, the patch 100 may include a plurality of third sensors 112. The patch 100 can include one, two, three, or more third sensors 112. In embodiments in which patch 100 includes multiple third sensors 112, the third sensors 112 can be arranged at different spatial positions on the base 102 relative to each other or relative to other components of the patch 100. For example, the third sensors 112 can be arranged in a third geometric arrangement 114. Such arrangements can supply the patch 100 with spatial oxygen concentration data at different points in time. This data can be used to track aspects of the physiological response. For example, this data can be used to track the spread of swelling over time. As shown in FIG. 1, the patch 100 can include two third sensors 112—one provided in each of the first zone 102a and the second zone 102b of base 102. The third sensors 112 can be arranged in any suitable number of zones of the patch 100. The third geometric arrangement 114 can include any suitable arrangement of the third sensors 112 relative to one another, such as 1 by 1, 2 by 1, 3 by 1, 3 by 2, 3 by 3, or another suitable arrangement. The third geometric arrangement 114 may be disposed adjacent to, or relative to, a hole 103 extending through the base 102. The third geometric arrangement 114 may be disposed radially around the hole 103.

In some embodiments, the patch 100 can include a controller 116. The controller 116 can include a printed circuit board assembly having a processor with a non-transitory computer readable medium (e.g., memory) that can include instructions for controlling any of the sensors included in patch 100. In addition, the controller 116 can include an input/output (e.g., a receiver, transmitter, etc.) for exchanging data with an external source 118 (e.g., a smartphone, a network, or any other device capable of digitally receiving data).

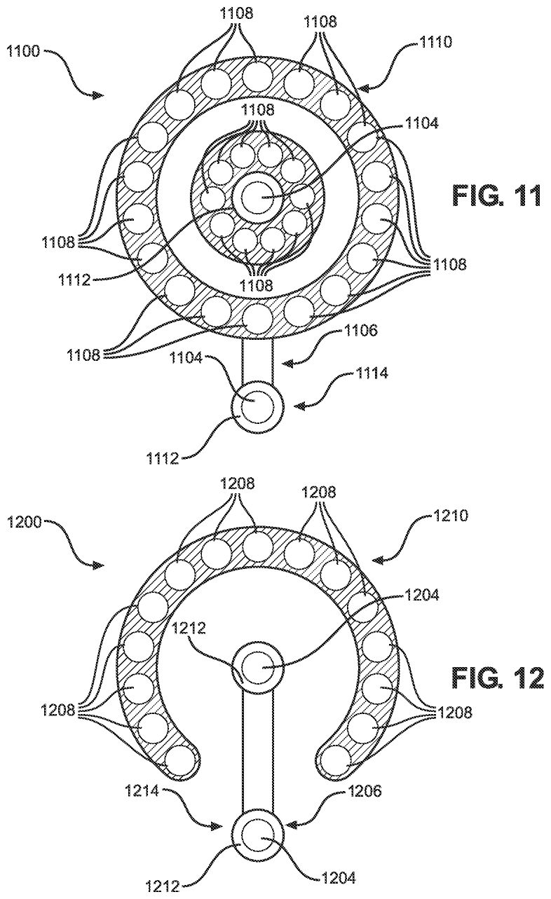

FIGS. 11, 12, 13, 14, and 15 show patches 1100, 1200, 1300, 1400, and 1500, respectively, that can be configured to sense multiple distinct conditions indicative of a physiological response. For example, as shown in FIG. 11 the patch 1100 can include a first geometric arrangement 1106 having one or more first sensors 1104, a second geometric arrangement 1110 having one or more second sensors 1108, and a third geometric arrangement 1114 having one or more third sensors 1112. The first geometric arrangement 1106 and the third geometric arrangement 1114 can respectively include two first sensors 1104 and two third sensors 1112 arranged in a 2 by 1 geometric arrangement. The third sensors 1112 of the third geometric arrangement 1114 can be provided concentrically around the first sensors 1104 of first geometric arrangement 1106. The second geometric arrangement 1110 can include a plurality of second sensors 1108 arranged in concentric circles. The second geometric arrangement 1110 can be centered around one of the groupings of the first sensors 1104 and the third sensors 1108 of first geometric arrangement 1106 and third geometric arrangement 1114, respectively.

As schematically shown in FIG. 12, the patch 1200 can include a first geometric arrangement 1206 having one or more first sensors 1204, a second geometric arrangement 1210 having one or more second sensors 1208, and a third geometric arrangement 1214 having one or more third sensors 1212. The first geometric arrangement 1206 and the third geometric arrangement 1214 can respectively include two first sensors 1204 and two third sensors 1212 arranged in a 2 by 1 geometric arrangement, as represented schematically in FIG. 12. One each of the first sensor 1204 and the third sensor 1212 can be disposed towards a geometric center of patch 1200, while another one of the first sensor 1204 and the third sensor 1212 can be disposed at a distance away from the geometric center of patch 1200. The second geometric arrangement 1210 can include a plurality of second sensors 1208 arranged in a horseshoe-shape around the center of the patch 1200.

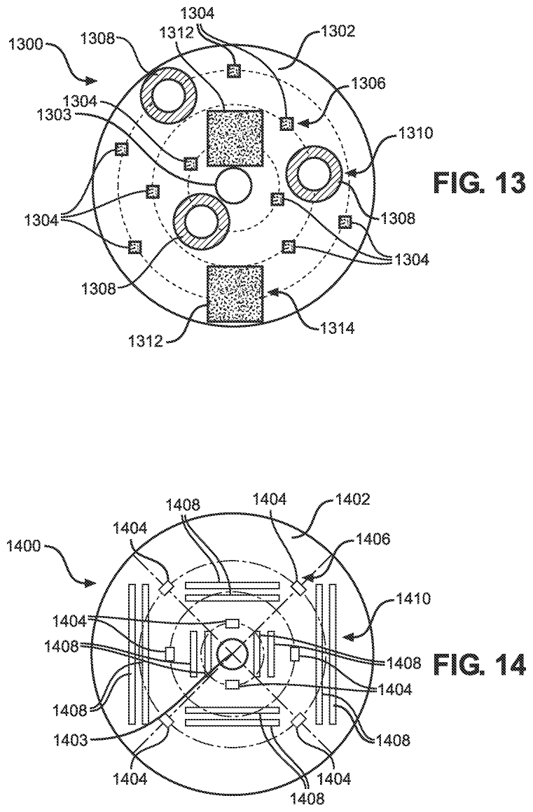

As shown in FIG. 13, the patch 1300 can include a first geometric arrangement 1306 having one or more first sensors 1304, a second geometric arrangement 1310 having one or more second sensors 1308, and a third geometric arrangement 1314 having one or more third sensors 1312, which can be provided on a circular base 1302 that can have a hole 1303. The first geometric arrangement 1306 can include a plurality of first sensors 1304 distributed throughout zones of the base 1302. The first geometric arrangement 1306 can include, for example, nine first sensors. The second geometric arrangement 1310 can include a plurality of second sensors 1308 distributed throughout the zones of the base 1302. For example, the second geometric arrangement 1310 can include three second sensors 1308 arranged in a triangular geometric arrangement. The third geometric arrangement 1314 can include a plurality of third sensors 1312 distributed throughout the zones of the base 1302. For example, the third geometric arrangement 1314 can include two third sensors 1312 arranged in a 2 by 1 geometric arrangement.

As shown in FIG. 14, the patch 1400 can include a first geometric arrangement 1406 and a second geometric arrangement 1410, which can be provided on a circular base 1402 that can have a hole 1403 extending therethrough. The first geometric arrangement 1406 can include a plurality of first sensors 1404 distributed throughout the zones of the base 1402. In some aspects, the first geometric arrangement 1406 can include eight first sensors 1404. The second geometric arrangement 1410 can include a plurality of second sensors 1408 distributed throughout the zones of the base 1402. In some aspects, the second geometric arrangement 1410 can include six second sensors 1408 arranged in parallel bars that can be oriented about the geometric center of the patch 1400. In embodiments where the patch 1400 defines a hole 1403 therethrough, the second sensors 1408 can be arranged around the hole 1403.

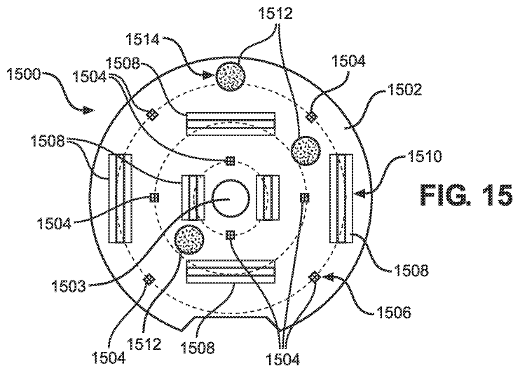

As shown in FIG. 15, the patch 1500 can include a first geometric arrangement 1506 having one or more first sensors 1504, a second geometric arrangement 1510 having one or more second sensors 1508, and a third geometric arrangement 1514 having one or more third sensors 1512, which can be provided on a circular base 1502 that can have a hole 1503. The first geometric arrangement 1506 can include a plurality of first sensors 1504 distributed throughout the zones of the base 1502. In some aspects, the first geometric arrangement 1506 can include eight first sensors 1504. The second geometric arrangement 1510 can include six second sensors 1508 arranged in arranged in parallel bars that can be oriented about a geometric center of the patch 1500, which can include a hole 1503. The third geometric arrangement 1514 include three third sensors 1512 arranged in a triangular geometric arrangement. The base 1502 can include an indentation for accommodating an external device, such as a USB cable. The patches 1100, 1200, 1300, 1400, and/or 1500 can include any of the features of any of the previously described patches, and vice versa.

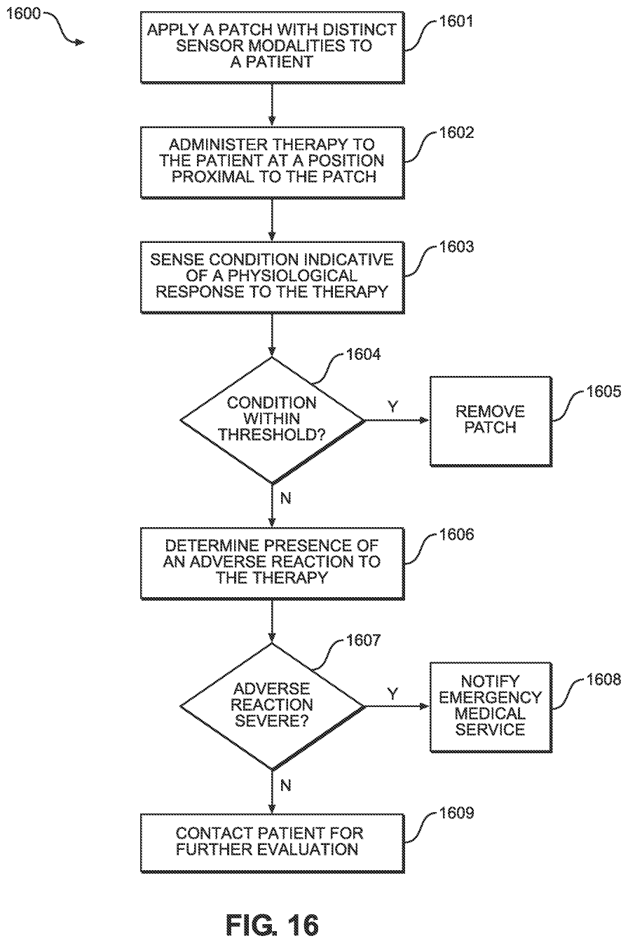

FIG. 16 shows a process 1600 for using any of the above-described patches to automatically determine a presence of an adverse reaction to a therapy. The process 1600 can include, at step 1601, applying any of the above-described patches to a patient. In some embodiments, the process 1600 can include applying a patch with two distinct sensory modalities to the patient.

The process 1600 can include, at step 1602, administering a therapy to the patient at a position proximal to the patch applied at step 1601. In some embodiments, the therapy can be self-administered, where the patient administers the therapy to him or herself. Alternatively, the therapy can be administered by a separate party, such as a healthcare provider. In some embodiments, the therapy can include injecting a treatment (e.g., a medicament) into the patient. The injection can be performed through a hole in the patch.

The process 1600 can include, at step 1603, sensing, with at least one of the sensors of the patch, a condition indicative of a physiological response. The condition can be any of the above-discussed conditions, including for example temperature, impedance, and oxygen concentration. The physiological response can be, for example, swelling.

The process 1600 can include, at step 1604, comparing the condition sensed at step 1603 with a threshold value for that condition. The comparison can be automatically performed by a controller of the patch. If the condition sensed at step 1603 is within an acceptable range of the threshold value for that condition, the process 1600 can proceed to step 1605 and the patch can be removed from the patient. Alternatively, if the condition sensed at step 1603 is outside of the acceptable range of the threshold value for that condition, the process 1600 can proceed to step 1606 and determine the presence of an adverse reaction to the therapy.

In response to determining the presence of an adverse reaction to the therapy at step 1606, the process 1600 can include, at step 1607, evaluating the severity of the adverse reaction. For example, if the condition sensed at step 1603 is outside of an elevated range of the threshold value for that condition, the adverse reaction can be considered severe. The elevated range can be a higher value or lower value than the threshold signifying a greater risk of the condition to the patient. If the condition sensed at step 1603 is within the elevated range of the threshold value for that condition, the adverse reaction would not be considered severe. This evaluation can be automatically performed by a controller of the patch.

In response to determining that the adverse reaction is severe at step 1607, the process 1600 can include, at step 1608, contacting emergency medical services. For example, a controller of the patch can automatically and directly or indirectly initiate a communication to emergency services providing information about the patient (e.g., location, severity of reaction, etc.) and requesting immediate assistance.

In response to determining that the adverse reaction is not severe at step 1607, the process 1600 can include, at step 1609, contacting the patient for further evaluation. This could include, for example, the controller of the patch automatically and directly or indirectly initiating a communication to the patient encouraging them to seek medical attention for the adverse reaction. Additionally, or alternatively, the controller of the patch directly or indirectly could initiate a communication to a healthcare provider for further evaluation and discussion with the patient.

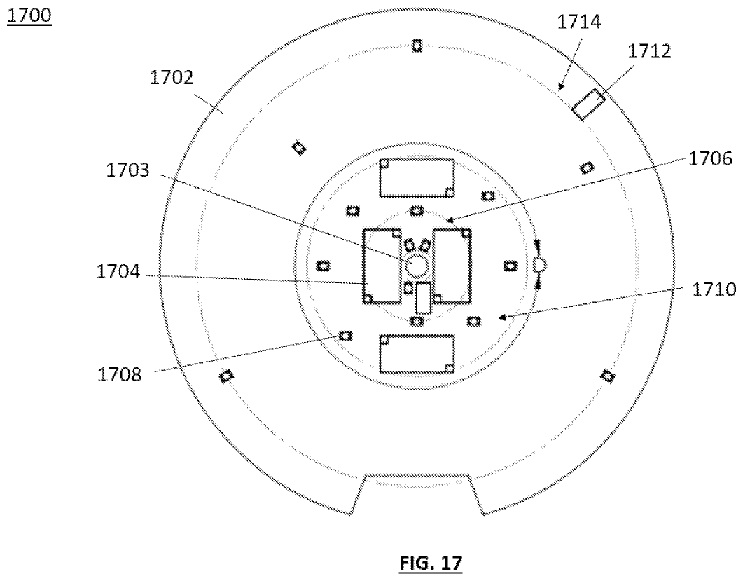

A patch 1700 is depicted in FIG. 17. The patch 1700 may be similar to any other patch embodiments described herein and depicted in the figures. The patch 1700 has a base 1702 configured to contact the skin of a user. The base 1702 may include an adhesive. The patch 1700 includes a hole 1703 extending therethrough, the hole 1703 being configured to receive a needle therethrough. The patch 1700 may include a septum covering at least a portion of the hole 1703, the septum being pierceable by a needle. A plurality of sensors may be disposed on the base 1702. The patch 1700 can include a first geometric arrangement 1706 having one or more first sensors 1704, a second geometric arrangement 1710 having one or more second sensors 1708, and a third geometric arrangement 1714 having one or more third sensors 1712. In some aspects, one or more of the plurality of sensors may protrude from the surface of the base 1702, such that when the patch 1700 is placed on the skin, the protruding sensors contact the skin between the skin and the base 1702.

The base 1702 may be deformable to permit attachment to the skin in different configurations. In some aspects, the base 1702 may be placed flat on the skin, such that the entirety of the skin-contacting surface of the base 1702 contacts the skin. In other aspects, the base 1702 may be deformed or folded such that at least one portion of the base 1702 contacts the skin while another portion of the base 1702 is spaced from the skin. In such aspects, the base 1702 may be positioned to contact the skin along the circumference of the base 1702, while a portion of the base 1702 radially inward from the circumference may be spaced from the skin in the shape of a “tent” to define a pocket or gap between the base 1702 and the surface to which the patch 1700 is adhered. The protruding sensors may contact the skin and prevent the portion of the base 1702 from which the sensors protrude from contacting the skin, thus forming the “tent” shape. In some aspects, a tent shape may be advantageous by trapping air between the base 1702 and the skin, thus preventing free flow of air around the sensors, which could in turn result in an inaccurately sensed parameter (e.g., an inaccurate temperature). It will be understood that the base of any of the depicted patch embodiments described herein can be deformable as described above.

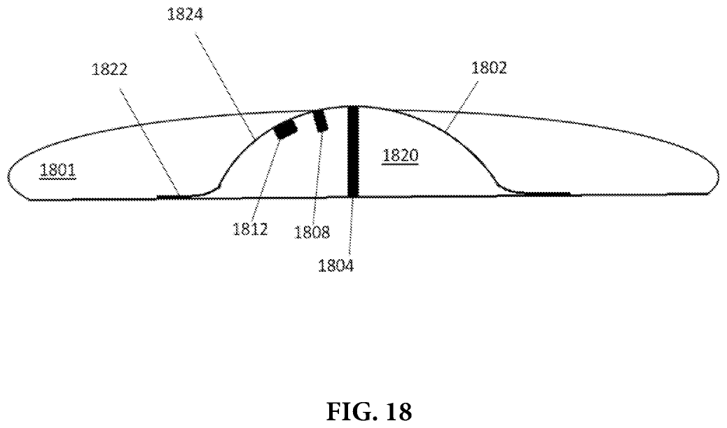

FIG. 18 depicts a cross-sectional view of a patch 1800 disposed on skin 1801. The patch 1800 is adhered to the skin via a base 1802. The base 1802 can be deformable as described above, or, alternatively, can be substantially rigid and manufactured to have a desired shape. The shape can be substantially flat (relative to the surface of the skin) as described in some of the other embodiments herein. In some aspects, the shape can be concave as depicted in FIG. 18 so as to define a pocket 1820 within the base 1802. The base 1802 may partially contact the skin 1801. When the patch 1800 is adhered to the skin 1801, a first portion 1822 of the base 1802 may be configured to contact the skin 1801, while a second portion 1824 of the base 1802 that is adjacent the first portion 1822 may be configured to be spaced from the skin 1801. In some aspects, the first portion 1822 may extend circumferentially around the base 1802, such that all or most of the circumference of the base 1802 contacts the skin 1801. The second portion 1824 may be radially adjacent to the first portion 1822 toward the geometric center of the base 1802.

The patch 1800 can include one or more sensors therein as described in the various embodiments in this application. FIG. 18 depicts a first sensor 1804, a second sensor 1808, and a third sensor 1812. In some aspects, at least one of the first, second, or third sensors 1804, 1808, or 1812 can be configured to extend from the second portion 1824 of the base 1802 into the pocket 1820. In some aspects, one or more of the first, second, or third sensors 1804, 1808, or 1812 can be configured to contact the skin 1801 when the patch 1800 is placed on the skin 1801. This contact may allow for a deformable base 1802 to elastically deform around these one or more sensors so as to form the pocket 1820 to be defined in the space between the skin 1801, the first portion 1822 of the base, and the second portion 1824. The presence of the pocket can be advantageous in trapping air or other gas therein to prevent undesired air flow past the one or more sensors to improve the accuracy of the sensor readings. It will be appreciated that the depiction of the first sensor 1804, second sensor 1808, and third sensor 1812 in FIG. 18 is exemplary and is not intended to limit the arrangement, quantity, or type of sensor. Aspects of the patch 1800 can include any combination of sensors in any geometric arrangement as described elsewhere in this application.

It will be appreciated that the foregoing description provides examples of the disclosed machine. However, it is contemplated that other implementations of the invention may differ in detail from the foregoing examples. All references to the invention or examples thereof are intended to reference the particular example being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated. All methods described herein can be performed in any suitable order unless otherwise indicated herein or otherwise clearly contradicted by context.

What is claimed is:

1. A patch for sensing conditions indicative of a physiological response, the patch comprising:
a base configured to be attached to a region of skin undergoing the physiological response;
a first sensor provided on the base that is configured to detect a first condition indicative of the physiological response of the region of skin;
a second sensor provided on the base that is configured to detect a second condition indicative of the physiological response of the region of skin; and
a third sensor provided on the base that is configured to detect a third condition indicative of the physiological response of the region of skin,
wherein the first sensor, the second sensor, and the third sensor are distinct sensor modalities that detect distinct conditions.

2. The patch of claim 1, wherein at least one of the first sensor, the second sensor, and the third sensor comprises two sensors disposed at different locations on the base.

3. The patch of claim 1, wherein the first sensor is a temperature sensor, and the first condition is a temperature of the region of skin.

4. The patch of claim 1, wherein the second sensor is an impedance sensor, and the second condition is an impedance of the region of skin.

5. The patch of claim 1, wherein the third sensor is an oxygen sensor, and the third condition is an oxygen concentration of the region of skin.

6. The patch of claim 5, wherein the oxygen sensor comprises a material that is phosphorescent in a presence of oxygen.

7. A patch for sensing a temperature and an impedance indicative of a physiological response, the patch comprising:
a base configured to be attached to a region of skin undergoing the physiological response;
a temperature sensor provided on the base that is configured to detect the temperature of the region of skin that is indicative of the physiological response; and
an impedance sensor provided on the base that is configured to detect the impedance of the region of skin that is indicative of the physiological response.

8. The patch of claim 7, wherein the temperature sensor comprises a first temperature sensor provided on an inner zone of the base and a second temperature sensor provided on an outer zone of the base extending radially from the inner zone of the base.

9. The patch of claim 7, wherein the temperature sensor is a digital integrated circuit sensor.

10. The patch of claim 7, wherein:
a first portion of the base contacts the skin;
a second portion of the base adjacent the first portion is spaced away from the skin;
a pocket is defined between the second portion of the base and the skin; and
at least one of the temperature sensor or the impedance sensor extends from the base into the pocket.

11. The patch of claim 7, wherein the temperature sensor comprises a resistive thermal difference element.

12. The patch of claim 7, wherein the impedance sensor comprises a first impedance sensor provided on an inner zone of the base and a second impedance sensor provided on an outer zone of the base extending radially from the inner zone of the base.

13. The patch of claim 7, wherein the impedance sensor comprises a wet electrode or a dry electrode.

14. A method for automatically determining an adverse reaction to a therapy, the method comprising:
applying a patch to a patient, the patch comprising a first sensor modality and a second sensor modality that are each configured to detect distinct conditions indicative of a physiological response of the patient;
administering the therapy to the patient at a position proximal to the patch;
sensing a condition of the distinct conditions indicative of the physiological response with at least one of the first sensor modality and the second sensor modality;
comparing the condition to a threshold; and
determining, based on comparing of the condition to the threshold, the adverse reaction to the therapy.

15. The method of claim 14, further comprising evaluating, in response to determining the adverse reaction, a severity of the adverse reaction by comparing the condition to an elevated range of the threshold.

16. The method of claim 15, further comprising determining, in response to comparing the condition to the elevated range of the threshold, that the adverse reaction is severe.

17. The method of claim 16, further comprising automatically contacting emergency services in response to determining that the adverse reaction is severe.

18. The method of claim 15, further comprising determining, in response to comparing the condition to the elevated range of the threshold, that the adverse reaction is not severe.

19. The method of claim 14, wherein the therapy includes a self-administered injection.

20. The method of claim 14, wherein the physiological response is swelling.
